(19) United States
(12) Patent Application Publication
(10) Pub. No.: US 2019/0357560 A1
(43) Pub. Date: Nov. 28, 2019

(54) COMPOSITIONS FROM CACAO PERICARP AND METHODS OF PRODUCING AND USING THEM

(71) Applicant: Guillermo Ceballos, Tijuana (MX)

(72) Inventor: Guillermo Ceballos, Tijuana (MX)

(21) Appl. No.: 16/538,659

(22) Filed: Aug. 12, 2019

Related U.S. Application Data
(63) Continuation-in-part of application No. PCT/MX2018/000012, filed on Feb. 13, 2018.
(60) Provisional application No. 62/458,408, filed on Feb. 13, 2017.

(30) Foreign Application Priority Data
Aug. 9, 2019 (MX) MX/A/2019/009553

(51) Int. Cl. A23G 1/48; A61K 8/73; A61K 8/67; A23L 33/00; A23G 1/50

(52) U.S. Cl. CPC A23G 1/48; A61K 8/735; A23G 1/50; A23L 33/30; A61K 8/676

(57) ABSTRACT

Cacao pod pericarp (CP) is used to produce a food ingredient, or flour, using convective-heat or freeze-drying and/or microwave drying methods. The observed physical and technological properties of dried CP revealed its potential application as an ingredient in manufactured food products. The levels of bioactive compounds are significantly increased in the dried CP samples compared to the raw material, especially in the case of the freeze dried methods. The commercially advantageous use of the cacao pod waste material can be used in treatments for humans and animals in order to improve several anthropometric measurements or cardiometabolic markers in the blood and to improve the composition and mass of the body. Embodiments for treating humans and animals include administering orally to decrease LDL/HDL ratios or triglyceride/HDL ratios, or administering topically as a cosmetic to treat fine lines, wrinkles, skin discoloration, and other skin conditions.

REFERENCE TO RELATED APPLICATIONS

This application claims priority to U.S. provisional patent application 62/458,408, filed Feb. 13, 2017, the entire contents of which are incorporated herein by reference. This application is filed under 35 U.S.C. § 111(a) as a continuation-in-part of PCT/MX2018/000012, filed Feb. 13, 2018, which application designates the United States. Applicant also claims priority to Mexican national stage application MX/a/2019/009553, filed Aug. 9, 2019.

### FIELD OF THE INVENTION AND INTRODUCTION

The invention relates to economically advantageous uses of the pericarp from the pod of the Theobroma cacao plant and comestible products and compositions derived from this material. The pericarp from the cacao pod is typically a waste product in cacao bean production and the methods described here make use of what formerly would be discarded cacao material. In particular, the invention employs the pericarp of the pod and its commercial use in producing a dietary supplement or food ingredient. Also, as shown here, methods of the invention result in compositions that possess unexpectedly high levels of beneficial compounds, resulting in products and treatments for reducing blood triglyceride levels, for reducing LDL levels, for reducing blood glucose levels, for weight maintenance and loss, for reducing waist circumference, for improving muscle strength, and for improving other cardiometabolic markers in the blood. In another aspect, the pericarp material can be used to make a topical cosmetic composition, and in one example a topical cream, that can treat skin discoloration, wrinkles and fine lines, and other condition of the skin.

### BACKGROUND

Theobroma cacao pericarp (CP) is an agricultural by-product generated in abundance on plantations and farms when mature cacao seeds or beans are removed for bean processing. The term CP is used here because it is a specific term defined by in plant biology and refers to the outer tissues of the cacao pod. Cuatrecasas, J., Cacao and Its Allies: A Taxonomic Revision of the Genus Theobroma, Contributions from the United States National Herbarium (Washington DC) 35, part 6. 1964. The pericarp surrounds the cacao seed and the surrounding seed pulp. Other casual terms that may be found in the prior art may include cacao hull, cacao shell and cacao pod, but these terms are confusing and frequently are used to refer to structures of the cacao beans, such as the seed coat. In the chocolate industry, the seed coat is part of whole cacao beans that has been typically fermented and dried. This seed material is not pericarp. The seed material can, and often is, referred to as shell, cacao bean husk, pod, husk or hull material in scientific literature and patent documents. The seed coat is removed from cacao seed either before or after bean roasting and before chocolate manufacture begins. So to avoid confusion the term CP (Theobroma cacao pericarp) will be used in this invention.

The CP is a major waste product of the cacao bean production on farms or plantations, and hence for the cacao bean production industry (Yapo, B. M. et al. Adding value to cocoa pod husks as a potential antioxidant-dietary fiber source. Am. J. Food Nutr. 1, 38-46. 2013 Karim et al., Phenolic composition, antioxidant, anti-wrinkles and tyrosinase inhibitory activities of cocoa pod extract. BMC complementary and alternative medicine, 14, 381 (2014). According to the International Cocoa Organization (ICCO), 4.4 million tons of cacao beans were produced during 2013-2014, and approximately 3 million tons of CP were discarded on farms and plantations, causing environmental problems, foul odors, and propagating diseases, such as black pod rot (ICCO 2015, Karim et al., 2014, Vriesmann et al., 2011, Vriesmann et al., 2012).

In Mexico and other cacao-producing countries, the processing of cacao pericarp (CP) into useful byproducts could provide economic advantages to farmers and reduce environmental problems (Vriesmann et al., Cacao pod husks (Theobroma cacao L.): composition and hot-water-soluble pectins. Industrial Crops and Products, 34, 1173-1181 (2011). CP is rich in a variety of compounds, making it a promising source of such molecules; thus, could hold potential for the production of useful natural compounds. The total phenolic content of cacao pod husk is 46.4±0.04 mg of gallic acid equivalents/g, demonstrating that it is a rich source of phenolic compounds that could be used rather than discarded (Karim et al., 2014).

However, conserving the phenol content of CP is challenging because this material decomposes rapidly, especially in hot, moist, humid tropical climates. In addition, the location of most cacao plantations does not generally allow for local storage or preservation methods. Perishable fruits, vegetables and other agro-industrial by-products can be dehydrated to preserve them for subsequent use (Si et al., Comparison of different drying methods on the physical properties, bioactive compounds and antioxidant activity of raspberry powders. Journal of the Science of Food and Agriculture (2015)). Dehydration extends the shelf life of the final food product, allowing its convenient long-term storage and future use in the manufacture of food, beverages or supplements. Among the different drying processes that are utilized, hot-air drying is commonly used in food production because it is an affordable method, although the long drying period can result in an inferior product quality.

In addition, the use of high temperatures for dehydration is controversial because naturally bioactive compounds are relatively unstable under heat and, thus, nutrients can be lost during thermal processing (Choi et al., 2006). Conversely, studies have shown that the phytochemical content and antioxidant and biological activities of processed foods may be increased by heat treatment causing degradation of cellular constituents, thus promoting the release of phytochemicals into media (Choi et al., 2006, Wojdylo et al., 2009).

Although various methods and conditions for drying are presumed to differentially affect the quality and quantity of the bioactive compounds present in fruits and vegetables, there is no information regarding the effect of using drying techniques on the bioactive compounds present in CP. Furthermore, nothing is known about the beneficial effect of such methods on the polyphenol or antioxidant content in these materials when consumed by humans.

The invention, in part, addresses these shortcomings and provides compositions produced from CP material that can be used for human and animal consumption. As explained below, the methods of the invention can be used to produce a food supplement or a food ingredient that, when consumed regularly or daily, advantageously leads to a dramatic reduction in blood triglyceride levels, and beneficial improvement in both LDL and HDL levels. Furthermore, methods of treating subjects with the CP food supplement or ingredient result in weight loss and improvements in muscle strength. In another aspect, the CP material is used to make a topical cosmetic composition or a topical cream that can be used treat skin discoloration, wrinkles, fine lines, or other conditions of the skin.

### BRIEF SUMMARY OF THE INVENTION

In one aspect, the invention provides a new, rich source of beneficial cacao-derived compounds, such as antioxidants, polyphenols, and flavonoids. In another aspect, this invention provides methods of treating humans and animals to improve the levels of several blood markers associated with disease and/or improved health. In another aspect, the invention encompasses commercially advantageous methods of producing a food supplement, ingredient or functional food made at least in part from the cacao pod husk pericarp material. The “pericarp” is distinguished by three layers: the exocarp or outer skin of the pod; the thick white mesocarp just below the exocarp; and a thin translucent inner layer called the endocarp (see FIG. 1). Collectively these layers comprise the outer part of the cacao fruit or pod and are collectively referred to as the pericarp. The cacao seeds or beans and the outer layer or membrane surrounding the seeds are distinct from the pericarp. The seeds or beans are also surrounded in the pod by a mucilaginous pulp material, which again, are distinct from the pericarp. Cacao nibs, the part of the seed used in chocolate manufacture, are surrounded by the cacao shell, and the nibs and the cacao shell are not part of the pericarp.

In a more specific aspect, the invention includes a method for the preparation of a “flour” food ingredient or composition from CP. The method comprises isolating pericarp material from cacao pods, where the pericarp material is free of cacao seeds and pulp, forming a paste and drying the pericarp paste, and then grinding the dried material into a flour-type product or processing it into another food ingredient, such as a powder, paste, or extract. In preferred embodiments, the method employs a freeze dying step under vacuum pressure until the pericarp material has a moisture level of 10% or less, or 8% or less. In another preferred embodiment, the method employs a microwave drying step until the pericarp material has a moisture level of 10% or less, or 8% or less, or 5% or less. Combinations of these and other drying methods can also be used. The pericarp material can be ground to obtain pericarp flour with a particle size of less than 0.500 mm, or less than 0.425 mm.

In any of the methods or the uses of the compositions described here, the invention can include measuring the total polyphenol content, total flavonoid content, and/or antioxidant capacity of the pericarp material or the flour composition or food ingredient. Thus, the invention allows the production of compositions and the monitoring of the advantageously high levels of beneficial cacao-derived flavonoids and polyphenols in those compositions.

In another aspect, the invention encompasses methods for enhancing the antioxidation capacity of cacao pod husk material comprising isolating pericarp material from cacao pods after harvesting cacao seeds from the pods so that the pericarp material is substantially free of cacao seeds and pulp material, then drying the pericarp material to a moisture level of 10% or less, or 8% or less. This drying process substantially concentrates the solid and non-water compounds that occur in the pericarp. The dried material is then ground to form a flour composition or other food ingredient. The method of drying can be selected from one or more of freeze drying under vacuum, oven heating, and microwave treatment. The methods can further comprise measuring the polyphenol oxidase activity of the pericarp material or the flour composition or food ingredient.

In another aspect, the invention includes prophylactic methods and methods of treating a human subject and/or animals by providing a CP ingredient derived only from the pericarp of the pod, and administering the ingredient daily or multiple times per day. For human subjects in particular, the amount delivered daily will optimally include enough of the pericarp-derived cacao composition to deliver about 12.5 mg of total cacao flavonoids per dose. Trace amounts of other cacao pod material can be present in any of the compositions of the invention without departing from the “derived only from pericarp” recitation or any similar construction used herein, as production methods and pericarp collection procedures may not be exact in all cases. However, the preferred embodiments exclude all other cacao pod materials. Other amounts, dosages, and administration regimens can also be used and adopted. In embodiments of the invention related to methods of treating animals or humans, or methods of treating or preventing disease in humans or animals, the antioxidant capacity of cacao-derived material improves physiological markers associated with certain disease conditions. For example, one or more of the following markers or measurements can be used: triglyceride levels; LDL levels; glucose levels; LDL/HDL ratio, Body Mass Index; muscle mass; or triglyceride/HDL ratio. Accordingly, daily treatments with beneficial cacao-derived compounds, or the cacao pod husk pericarp flour described here, can advantageously improve health or prevent disease.

In other aspects, the methods for preparing ingredients and compositions can result in a flour-like composition, a paste, and/or an extract of CP. Various extraction methods are known in the art and can include the use of food-approved chemicals and solvents. Administering the ingredients or compositions may be done with any suitable carrier, in solid or liquid dosage form such as tablets, capsules, powders, soft gels, solutions, suspensions, emulsions, ointments, or creams. The ingredient or composition may also be administered as a food supplement or as a functional food ingredient. An extract of the pericarp material or flour may be used alone or combined with one or more other plant extracts to produce a composition optimized for a particular functional effect. Further, an extract composition may be formulated with one or more other dietary nutrients, such as vitamins, minerals, amino acids, etc., to provide a functional or nutritional supplement for a desired health effect. These ingredients may be combined with necessary binders, excipients, preservatives, colors and the like known to those in the industry or commonly used or described in similar products in order to produce a suitable tablet, capsule, pill, liquid, cream, powder, or food ingredient. As noted, some of these ingredients or compositions can also be used for topical treatments or used in cosmetic products.

In another aspect, the CP flour composition can be used in a topical cosmetic composition, and in one example a topical cream. The various CP compositions described herein can be blended with a cosmetically acceptable carrier, which may include purified water, oils, alcohols, glycols, other cosmetically acceptable components available, and combinations thereof. The topical cosmetic compositions and the methods of using them employ the CP flour present in a range from approximately 0.1% to 1% by weight, or 0.05% to 1% by weight, or 0.05% to 5% by weight, for example.

In yet another aspect, the topical cosmetic composition may further comprise additional ingredients such as penetration enhancers, humectants, lubricants, pharmaceutically active agents, color agents, fragrance, preservatives, antioxidants, chelators, neutralizers, amino acids, anti-inflammatory agents, anti-irritants, anti-tack agents, astringents, binders, catalysts, stabilizers, emollients, emulsifiers, surfactants, cell-signaling agents, retinols, essential oils, plant/botanical extracts, conditioners, film formers, gelling agents, foaming agents, exfoliants, vitamins, minerals, pH adjusters, proteins, peptides, tactile enhancers, saccharides, solvents or any combination thereof.

In still another aspect, the topical skin care composition may be formulated as a cream, lotion, serum, facial cleanser, toner, eye cream, sunscreen, stick, spray, impregnated personal care device, impregnated towelette, gel, fluid/liquid, soap, oil, butter, peel, scrub, mask, concentrate, or any other form known in the art.

In another aspect, the present invention includes a topical skin care composition comprising a CP flour composition and a cosmetically acceptable carrier together with and one or more of the following: (a) a source of bio-retinol (b) a source of retinol-like activity; (c) a source of sodium hyaluronic acid or other cosmetically active acid; (d) a source of moisturizing saccharide complex; (e) an ascorbic acid source; and (f) a source of anti-oxidant components.

In still another aspect, a topical cosmetic composition in accordance with principles of the invention or the examples below is applied to the skin, such as the hands or face, which may have, for example, but not limited to, wrinkles, fine lines, uneven tone, loss of firmness, surface roughness, dark circles, under-eye puffiness, sun damage, redness, dryness, irritation, enlarged ports and combinations of all or some of the above. Alternatively, the topical cosmetic composition may be applied to the skin to prevent the occurrence of the various problems described above. Thus, the invention includes methods of treating and preventing skin conditions using a CP flour composition in a cosmetically acceptable carrier. Such methods include the daily use of the topical cosmetic composition on skin, and the use at multiple times during a day, for a period of time such as four weeks or more.

The invention is not limited by the foregoing, or the following Drawings, Description, Claims, or Examples, but can be appreciated in its full extent from the entire contents of this disclosure. None of the cited references or information should be taken to limit or modify any specific description or definition as used in this disclosure.

### DESCRIPTION OF THE DRAWINGS

The patent or application file contains at least one drawing executed in color. Copies of this patent or patent application publication with color drawings will be provided by the Office upon request and payment of the necessary fee. The following figures are examples of the scope and content of the invention and are not meant to limit the claims to any particular aspect or embodiment of the invention. FIG. 1 is a top view of a cross-sectional photograph of a Theobroma cacao pod showing the layers of plant matter and the relative location of the seeds, exocarp, mesocarp, and endocarp (Cuatrecasas, J. 1964). The “pericarp” is distinguished by three layers; the exocarp or outer skin; the thick white mesocarp just below the exocarp; and a thin translucent inner layer called the endocarp. Collectively these layers comprise the outer part of the fruit and are of maternal origin. The seeds are surrounded by a mucilaginous pulp material, and both the seeds and the pulp are not part of the pericarp. The cocoa seeds are an article of worldwide commerce. Cocoa seeds, also known as cocoa beans, are processed into chocolate liquor, chocolate, cocoa butter, and cocoa powder. Cocoa seeds are the result of fertilization of the flower ovule and pollen nucleus and are distinct from the pericarp. If taken from the coca pod and allowed to germinate, seeds will become the next generation of cacao plants.

FIG. 2 is a schematic showing a side cross-sectional view of a Theobroma cacao pod listing the location of the seeds, exocarp, mesocarp, and endocarp.

FIG. 3 shows the total phenolic content (TPC) and total flavonoid content (TFC) of fresh and dry CP flour prepared by different methods: OHD—oven heat drying; MWD—microwave drying; and FD—freeze drying. Total phenolic content is expressed as mg gallic acid equivalent/100 g sample dry mass (DM) and total flavonoid content is expressed as mg of (-)-epicatechin equivalent/100 g sample dry mass. Levels on graphs that list different letters are statistically significant (p<0.05).

FIG. 4 shows the changes in hand grip strength after 8 weeks of consumption of the placebo or the experimental cookie (galletas) treatment (placebo cookies and CP flour-containing cookies). There is a positive and significant increase in strength with regular treatment of CP flour.

FIG. 5 is a photograph illustrating an example of wrinkles noted in the hand dorsum of a volunteer before the application of a CP based skin cream and after 4 weeks of local application.

FIG. 6 summarizes changes noted in the total height of hand dorsum wrinkles noted in subjects exposed to 4 weeks of either a placebo or a CP based skin cream. As can be noted a statistically significant reduction in the total height of wrinkles is noted in CP treated subjects.

FIG. 7 summarizes changes noted in the color of the hand dorsum noted in subjects exposed to 4 weeks of either a placebo or a CP based skin cream. As can be seen a statistically significant reduction in the dorsum hand color is noted in CP treated subjects.

FIG. 8 is a photograph illustrating an example of the improvement of skin color of a volunteer before the application of a CP based skin cream and after 4 weeks of local application.

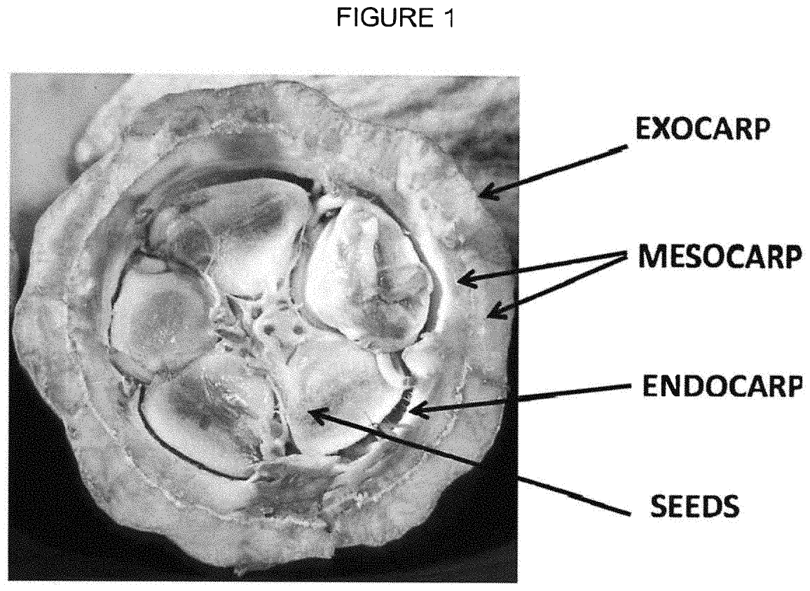

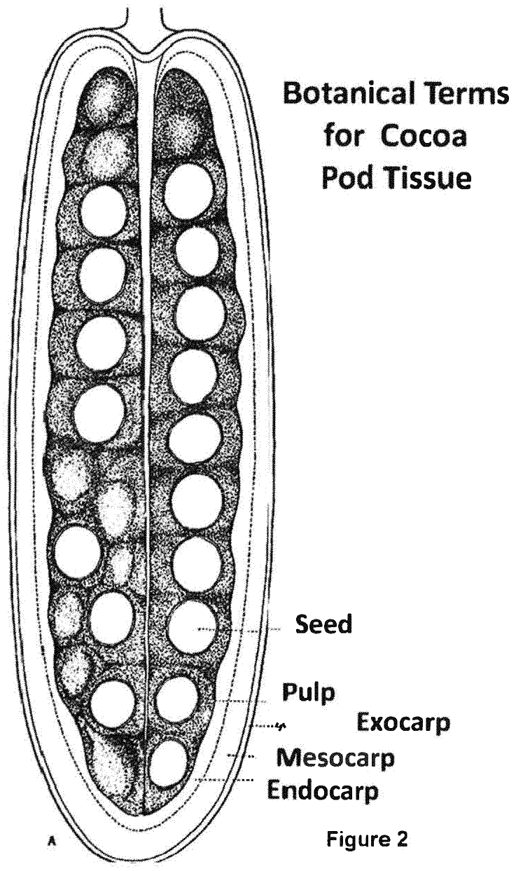

### DETAILED DESCRIPTION AND EXAMPLES

The invention in one aspect generally encompasses a method of producing and using a food ingredient from specific parts of the CP material. The ingredient, which can be formed as a flour for baking or food manufacture, can be used to produce edible and health-promoting products, such as wafers, baked cookies, cakes, and similar products. The ingredient can also be used as a supplement to be added to other food products or as a dietary supplement in pill or formulated form. The ingredient can also be used as an ingredient in creams or cosmetics to be applied to the skin.

As noted above, the preferred form for using the pericarp material is as a flour-type composition that is capable of being used as a food ingredient or in a food product, such as a baked food. However, the invention is not limited to this preferred form or use. In particular, the compositions or extracts of the CP material of the invention can also be used as a nutritional supplement. In such form, the compositions may be introduced into a base carrier or onto a carrier. The nutritional supplement may be enclosed within a capsule, typically a water soluble capsule, but could alternatively be water dispersible as a powder delivered from a sachet or a dispersible tablet, thus releasing the nutritional supplement once mixed with water. A capsule could also comprise one or more of a gum, such as acacia gum and Arabic gum, methyl cellulose gum, dextrin, gelatin, casein, milk powder, skimmed milk powder, soya protein, plant protein, or other protein. Alternatively, or in addition, the nutritional supplement can be carried on a granular, flake or particulate carrier, which again could be one or more of gum, such as acacia gum, gum Arabic, dextrin, methyl cellulose gum, gelatin, soya, plant protein, legume seed protein, soya milk, soya milk powder, soya protein, casein, vegetable gum, cellulose, starch, sugar, glucose, and/or maltodextrin.

Advantageously, a dietary supplement using the CP material can be provided in a caplet form for consumption one or several times per day. For instance, in one embodiment, the dietary supplement comprises a single caplet serving, each serving suitable for being consumed one, two, three or more times daily, e.g., at, before or between meals as appropriate.

In alternative embodiments, a pharmaceutical composition containing at least an extract of the cacao polyphenol of the pericarp material described herein, in an amount sufficient to achieve a desired effect in a subject, can be prepared. This composition may be a tablet, a liquid, capsules, soft capsule, paste or pastille, gum, or as a drinkable solution or emulsion, a dried oral supplement, or even a wet oral supplement. The pharmaceutical composition can further contain carriers and excipients that are suitable for delivering the biologically active compounds from cacao as well as other compounds of a different nature. The kind of the carrier/excipient and the amount thereof will depend on the nature of the cacao extract or cacao compounds or substance used and the desired mode of delivery and/or administration.

In addition, cosmetic or cosmeceutical compositions can be produced from the CP material described herein. A topical form can be a solution, emulsion, serum, skin and/or hair cleanser, body wash, body scrub, bar soap, liquid soap, shampoo lather, deodorant, skin and/or hair care preparation, foam, mousse, cream, lotion, pomade, balm, stick, gel, pump spray, aerosol spray, and combinations thereof. In one embodiment, the cosmeceutical compositions of this invention can be used in foams in personal care applications such as soaps, shampoos, skin cleansers, oral products, and the like. The compositions of this invention can thus also comprise a cosmetically acceptable carrier to act as a diluent, dispersant or carrier for the CP ingredients or extracts. The carrier can be one selected to facilitate the distribution and uptake of cacao-derived compounds when the composition is applied or administered. Vehicles other than or in addition to water can include liquid or solid emollients, solvents, humectants, thickeners, powders, and perfumes.

The following Examples and descriptions are exemplary only and should not be taken as a limitation on the scope of the invention.

### EXAMPLE 1

### Production of CP Flour

Step 1: Production of a CP Paste

Whole cacao pods are washed with soap, rinsed with water, sanitized for 15 min with Citrus21® solution (20 mL/L water, Integra Citrus, Ciudad de Mexico, Mexico). Pods were cut open, the seeds and pulp removed, and only the pericarp of the husk is retained and is ground in a semi-industrial blender (Crypto Peerless K55, USA) until a paste is obtained.

Step 2: Drying Methods of CP Paste

One or more of three methods can be used for drying of CP paste. Samples were dried using:

A. CONVENTIONAL HOT AIR OVEN (CD). 150 g of paste is dried at 60° C. for 24 h (Lindberg Blue OV-484, USA oven).

B. FREEZE DRYING (FD). 150 g of paste is dried under vacuum pressure of 20 Pa for 24 h (Scientz, SC-10N, Ningbo, China).

C. MICROWAVE DRYING (MWD). 150 g of paste is dried at microwave power (595 Watts) for 11.5 min using a Panasonic inverter NN-SN968, 2450 MHz.

Drying was conducted until the paste samples reached a 4-8% moisture level.

Step 3: Sifting and Milling of Dried Paste to Produce Cacao Pericarp Flour

Dried cacao pericarp paste is finely ground using a Cunill luxo mill and sieved to obtain:

Fine pericarp flour with a particle size≤0.425 mm.

Coarse pericarp flour with a particle size>0.425 mm.

### EXAMPLE 2

### Characterization of Pericarp Flour Samples

CP paste has a moisture content of 87.4±0.56%. The drying process reduces water content to 4.6-7.8% (see Table 1). The results indicate that when using freeze drying methodology, a lower moisture content was obtained compared to conventional and microwave paste drying. Moisture content of pericarp flour showed statistical differences (p<0.05) among the three drying methods used.

TABLE 1
Moisture, pH and water activity (A_{w})
of dried pericarp flour samples

| DRYING | MOISTURE | | |
|---|---|---|---|
| METHOD | PER 100 G | (A_{w}) | pH |
| CD | 5.98 ± 0.086 | 0.373 ± 0.003 | 5.57 ± 0.007 |
| MWD | 7.87 ± 0.38* | 0.543 ± 0.32* | 5.64 ± 0.02 |
| FD | 4.8 ± 0.34*^{,}** | 0.252 ± 0.018*^{,}** | 5.7 ± 0.014 |

*p < 0.05 vs. conventional,
**p < 0.05 vs. microwave

For pH determination, a suspension of 1 g of sample in 10 mL of distilled water is prepared with continuous stirring it for 2 min. The pH is determined using a pH meter (model pH 210, Hanna Instruments). Water activity of pericarp flour samples is measured using water activity meter Hygropalm α_{w} 1 (α_{w}-D10). Triplicate samples are measured at 23.6±2° C.

Water activity (Aw) and pH of dried pericarp flour are important parameters related to the prevention of product deterioration and degradation. The pH values were similar among the drying methods used resulting in a pH ranging from 5.57-5.7 (Table 1). Water activity of the dried pericarp flour had low values ranging from 0.252 (freeze drying) to 0.543 (microwave), showing a statistical difference among them (p<0.05) (Table 1). Interestingly, although microwave drying method presented the highest moisture and Aw values, the moisture and the Aw values are well below the safe storage upper limit conditions for the prevention of enzymatic and microbiological deterioration, indicating that any of the three drying methods can be used if only the preservation of the CP flour is considered.

Color assessment of these flour samples can be conducted at room temperature using a color reader (CR-10, Konica-Minolta Sensing Inc., Osaka, Japan). The parameters measured were L* for lightness; C* for chroma; A* for red; B* for yellow. Total color difference (ΔE) between fresh cacao and dried pericarp flour was determined using an equation. ΔE indicates the magnitude of color difference between fresh and dried samples. They can be analytically classified as slight difference (0.5>ΔE<1.5), noticeable difference (1.5>ΔE<3), marked difference (3>ΔE<6), extremely marked difference (6>ΔE<12), and a color of different shade (ΔE>12).

Color composition obtained from the three noted drying methods is presented in Table 2. The flour color evaluation showed that the cacao pericarp flour drying method used had significant effects. The main changes after drying were given in Hue* and b* values, having significantly increased compared to fresh pericarp sample indicating degradation of the original color by turning into more yellowish-brownish color. CP flour dried by three 3 different drying methods showed a wide ΔE from fresh pericarp sample (>20) indicating a total change of color shade, which can be appreciated at a glance. Freeze drying results in the greatest pericarp color change among the drying methods. The color results after drying treatments are important since color changes could affect the sensory properties of CP flour, limiting its utilization as a food ingredient, and also directly relates to changes in the composition of the separate samples.

TABLE 2
Color measurements of CP flour obtained using different drying methods.

| DRYING | | | | | HUE | |
|---|---|---|---|---|---|---|
| METHOD | A* | B* | C* | L* | ANGLE | ΔE |
| FRESH | 8.8 ± 1.2 | 10.2 ± 1.41 | 13.4 ± 1.9 | 36.1 ± 0.4 | 49.2 ± 0.1 | — |
| CD | 9 ± 0.1 | 17.3 ± 0.2* | 19.5 ± 0.3* | 54.9 ± 0.2* | 62.4 ± 0.1* | 20.2 ± 1.1* |
| MWD | 9.4 ± 0.2 | 19.4 ± 0.2* | 21.4 ± 0.2* | 56.2 ± 0.2* | 63.8 ± 0.1* | 22.2 ± 1* |
| FD | 11.97 ± 0.1* | 21.3 ± 0.1* | 24.4 ± 0.1* | 56.5 ± 0.1* | 60.7 ± 0.2* | 23.5 ± 1.2* |

Color difference (ΔE) is calculated using the fresh pericarp cacao flour (Fresh) as reference.
*= (p < 0.05) vs fresh.

Biochemical assessment of the CP flour extract can use total phenolic or flavonoid content determinations or the antioxidant activity from the method described by Martinez et al. (2012) with modifications. Five hundred milligrams of fresh and dry CP flours are stirred at room temperature for 60 min with 20 mL of acetone-water-acetic acid (70:29.5:0.5). These samples are centrifuged at 3,500 g, 15 min at 4° C. and the supernatants kept at −20° C. until used.

Total phenolic content (TPC) was determined using the Folin-Ciocalteu's reaction. A volume of 0.3 mL of the flour extracts is poured by triplicate into test tubes, adding 2.5 mL of Folin-Ciocalteu's reagent (diluted 1:10 with double distilled water) and 2 mL of sodium bicarbonate solution (7.5% w/v). Tubes are—rapidly stirred and mixed using a vortex stirrer, covered with parafilm and incubated at 50° C. for 5 min. Absorption at 760 nm is measured with a Genesys 10 UV spectrophotometer. Concentration is determined using gallic acid standard curves and results are expressed as mg of gallic acid equivalents (GAE)/g dry mass (mean±SD).

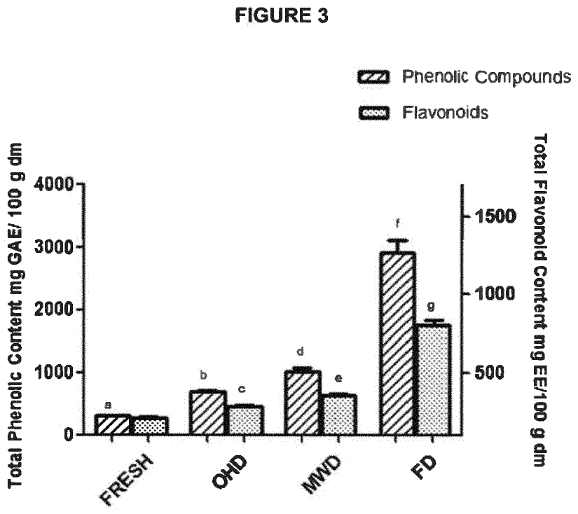

Total polyphenol content (TPC) of CP flour samples are shown in FIG. 3. Polyphenolic content was different (p<0.05) among the three drying methods used. The highest concentration was found in freeze-dried CP flour followed by microwave dried and the lowest by conventional oven drying. Freeze drying likely caused the retention of the most phenolic content since the method of drying does not use heat to dry samples, while with conventional oven drying, oxidative and thermal degradation or cross-link of polyphenols with pericarp structures probably increase given the long duration of the heat treatment. Interestingly, microwave drying process retained a considerable amount of phenolic compounds (a few mg below freeze drying) since it applies a short heating time, therefore, diminishing thermal degradation. Total flavonoid content (TFC) can be determined following the method described by Maleyki & Ismail, 2010. The TCF measurement is a non-specific and broad assessment of the total flavonoid content of a sample. This measurement alone does not imply or measure in vivo biological activity of the sample. Rather it is used to characterize a CPF sample lot. One milliliter of the cacao pericarp flour extract and 0.3 ml of NaNO₂ (0.5%) were added. The mixture is allowed to react by 5 min after adding 0.3 mL of AlCl₃ 6H₂O (10%). The mixture was stirred and allowed to react for 6 min. Two mL of 1 M NaOH were added and total volume adjusted to 10 mL with water. A UV spectrophotometer is used to read the absorbance at 510 nm. TFC is calculated with a calibration curve using (-)-epicatechin and expressed as mg (-)-epicatechin equivalents/g dry mass. Relative dosages in a single galleta or capsule or other preparation or supplement comprising CP flour can be calibrated by total polyphenol content equivalent to about 12 mg of (-)-epicatechin per dose/galleta/capsule or greater; or equivalent to 25 mg of (-)-epicatechin per dose twice a day or greater; or equivalent to a total flavonoids content of 12.5 mg or greater per day or greater; or equivalent to about a total flavonoids content of 12.5 mg for each of two treatments per day or greater. or equivalent to a total flavonoids content of 25 mg or greater per day or greater; or equivalent to about a total flavonoids content of 25 mg for each of two treatments per day or greater.

TFC results are also shown in FIG. 3. Flavonoid content was different (p<0.05) amongst the 3 drying methods used. The highest concentration was found in freeze-dried cacao pericarp flour followed by microwave dried and the lowest by conventional oven. Microwave drying process retained a considerable amount of flavonoids

Antioxidant activity can be determined by the radical scavenging properties of the flour samples, evaluated using the method previously reported by Brand-Williams et al, with some modifications. A stock solution is prepared by dissolving 24 mg DPPH (2,2-diphenyl-1-picrylhydrazyl) with 100 mL of methanol and then stored at −20° C. until needed. The DPPH solution is obtained by mixing 10 mL stock solution with 45 mL methanol until an absorbance of 1.1±0.02 units at 515 nm is obtained using the spectrophotometer. CP flour extracts (0.150 mL) were allowed to react with 2.85 mL of the DPPH solution for 2 h in the dark. Absorbance is measured at 515 nm using a UV-VIS spectrophotometer. The DPPH radical scavenging rate of samples was calculated using the following equation:

Inhibition %=[(A_blank−A_sample)/A_blank]×100

A_blank is the absorbance of solvent, and A_sample is the absorbance of the samples. The standard curve was linear between 25 and 800 μM of the synthetic antioxidant Trolox. Results are expressed in μM Trolox equivalent (TE)/g dry mass as mean of three replicates. It is important to also note that while ingredients may have inherent in vitro oxygen radical scavenging activity, this does not imply that such materials are healthful in vivo. This method is however useful in charactering an ingredient or a solution.

Results shown in Table 3 demonstrate that CP flour samples have the ability to scavenge DPPH free radicals. Among the three treatments, freeze drying and microwave methodologies show similar antioxidant potential, whereas conventional oven drying showed the lowest antioxidant capacity. However, any of the methods or indeed any combination of any of these drying methods can be selected and used. These results suggest that phenolic content may play an important role in determining antioxidant activity. Some polyphenols changes can occur during dehydration and could be related to the formation of new compounds or to the formation of polyphenols derivatives with higher antioxidant activity. CP flour displays dose-dependent antioxidant activity as per the DPPH assay (independently of method used to dry it) with a positive and significant correlation, freeze drying (r²=0.9786), conventional drying (r²=0.9561) and microwave drying (r²=0.9633).

For the ABTS (2,2′-azino-bis (3-ethylbenzothiazoline-6-sulphonic acid)) assay, the procedure followed the method described by Martinez et al., 2012. The stock solutions included 7.4 mM ABTS+ solution and 2.6 mM potassium persulfate solution. The working solution was then prepared by mixing the two stock solutions in equal quantities and allowing them to react for 12-16 h at room temperature in the dark. The solution is then diluted by mixing 1 mL ABTS+ solution with 60 mL methanol to obtain an absorbance of 1.1±0.02 units at 734 nm. CP flour extracts (150 μL) were allowed to react with 2850 μL of the ABTS+ solution for 2 h in a dark condition and then the absorbance was measured at 734 nm. The standard curve was linear between 25 and 800 μM of the synthetic antioxidant Trolox. Results are expressed in μM Trolox equivalents (TE)/g dry mass.

The ABTS assay results are shown in Table 3. Drying methods generated a significantly increase (p<0.05) in antioxidant activity with freeze drying of the CP flours which yielding the highest values. The higher antioxidant activity may be related to a better extractability of antioxidant compounds from the pericarp matrix using this method. As in the in vitro oxygen radical absorbance capacity, the in vitro Trolox equivalent assay is not meant to imply a biological activity in vivo. As reported here, this assay provides a guide to the relative levels of polyphenols and the antioxidant capacity as impacted by the processing of CP into shelf stable ingredients for quality control purposes.

TABLE 3
Antioxidant activity of fresh and dry CP flour
measured by the DPPH and ABTS methods.

| Sample | ABTS (μM TE/g dm) | DPPH (μM TE/g dm) |
|---|---|---|
| Fresh | 30.9 ± 1.24 | 17 ± 1.5 |
| OHD | 208 ± 7.70* | 47 ± 2.3* |
| MWD | 255 ± 10.9* | 48 ± 1.3* |
| FD | 317 ± 24.5* | 175 ± 14* |

In Table 3, OHD-oven heating drying, MWD-microwave drying, and FD-freeze drying.
The antioxidant activity was expressed as μM Trolox equivalent (TE) of dry mass (dm).
* = (p < 0.05) vs fresh.

### EXAMPLE 3

### Clinical Eight-Week Clinical Regimen Using Human Subjects

A placebo controlled clinical trial using a baked cookie food product can be used to demonstrate the beneficial effects of the pericarp flour consumed by humans and animals. Several clinical endpoints can be considered, including: body weight; body mass index (BMI); abdominal circumference; body fat; muscle mass; blood triglycerides (TG); blood glucose; blood cholesterol; blood LDL cholesterol; blood HDL cholesterol; TG/HDL ratio; LDL/HDL ration; and left hand grip strength.

Subject are recruited under the following criteria: Men or women from 20 to 60 years of age; BMI of >25 kg/m² to <29.9 kg/m²; and blood triglycerides 150-350 mg/dL. Subjects are excluded for any of the following: Type 2 diabetes mellitus; dyslipidemia; hypertension; cardiovascular diseases; subjects treated with hypolipidemic drugs, metformin or steroids; pregnancy or surgery in the last 6 months; lupus; arthritis; hypothyroidism; HIV or treatment with anti-retrovirals; alcohol or tobacco consumers; and a history of allergy to chocolate or components of cocoa.

Two groups, placebo/control and experimental, are recruited. Both groups (placebo and experimental, n=20 subjects per group) received nutritional counseling advising them to reduce 250 kilocalories/day from their usual diet and to perform moderate exercise so as to burn another 250 kilocalories/day.

After randomization to either group, the placebo group received control cookies (˜4 g and 14 Kcal) twice daily, one in the morning and one in the afternoon, before meals. Subjects in the experimental group received CP flour supplemented cookies (˜4 g and 14 Kcal) containing 12.5 mg of total flavonoids measured in equivalents of (-)-epicatechin, twice a day, one in the morning and one in the afternoon, before meals. Subjects, scientists and clinical laboratory technicians were blinded to treatment groups. Assaying the levels of flavonoids and epicatechin can be selected from the methods described in Jalil & Ismail 2008 (Polyphenols in cocoa and cocoa products: is there a link between antioxidant properties and health?. Molecules; 13(9): 2190-2219).

Baseline characteristics of recruited subjects are shown in Table 4. Both groups were equivalent at the beginning of 8-week trial.

TABLE 4
Baseline characteristics of placebo and experimental groups.

| | | GROUP B | |
|---|---|---|---|
| | | (CP | |
| | GROUP A | PERICARP | |
| | (PLACEBO) | FLOUR) | P |
| GROUP SIZE (N) | 20 | 20 | |
| | (MALE = 4 | (MALE = 3, | |
| | FEMALE = 16) | FEMALE = 17) | |
| AGE | 40.6 ± 2.4 | 48.3 ± 1.9 | 0.006 |
| BODY WEIGTH (KG) | 69.7 ± 2.2 | 66 ± 1.2 | 0.7 |
| BMI (KG/M²) | 27.8 ± 0.4 | 27.9 ± 0.3 | 0.3 |
| ABDOMINAL | 91.5 ± 1.3 | 87.9 ± 1.3 | 0.5 |
| CIRCUMFERENCE (CM) | | | |
| % BODY FAT | 35 ± 1.3 | 35.4 ± 0.6 | 0.7 |
| MUSCLE MASS (KG) | 43.4 ± 2.1 | 40.5 ± 1.02 | 0.1 |
| TRIGLYCERIDES (MG/DL) | 207.8 ± 15.8 | 189.8 ± 17.6 | 0.5 |
| GLUCOSE (MG/DL) | 84 ± 2.3 | 89.5 ± 3.1 | 0.2 |
| CHOLESTEROL (MG/DL) | 178.5 ± 7.9 | 204.8 ± 13.4 | 0.1 |
| C-LDL (MG/DL) | 98.8 ± 7.5 | 124.8 ± 11.1 | 0.07 |
| C-HDL (MG/DL) | 38.1 ± 1.8 | 42 ± 2.0 | 0.1 |
| TGL/HDL INDEX | 5.6 ± 0.5 | 4.9 ± 0.4 | 0.06 |
| LDL/HDL INDEX | 3.2 ± 0.2 | 3.0 ± 0.3 | 0.3 |

The results of placebo and pericarp flour treated subjects are shown in Tables 5 and 6.

TABLE 5
Anthropometric data after 8 weeks of cookie intake.

| | Group A | | | Group B | | |
|---|---|---|---|---|---|---|
| | placebo | | | Cacao Pericarp Flour | | |
| | Basal | Final | p | Basal | Final | P |
| Body weight (kg) | 69.7 ± 2.2 | 67.2 ± 2.3 | <0.001 | 66.0 ± 1.2 | 62.8 ± 1.1 | <0.0001 |
| BMI (Kg/m²) | 27.8 ± 0.4 | 26.8 ± 0.5 | <0.001 | 27.9 ± 0.3 | 26.6 ± 0.3 | <0.0001 |
| Abdominal | 91.5 ± 1.4 | 87.7 ± 1.7 | <0.001 | 87.9 ± 1.3 | 84.0 ± 1.6 | <0.0001 |
| Circumference | | | | | | |
| (cm) | | | | | | |
| % Body fat | 35 ± 1.3 | 33.7 ± 1.4 | <0.01 | 35.4 ± 0.6 | 35.0 ± 0.8 | 0.2 |
| Muscle mass (kg) | 43.4 ± 2.1 | 42.4 ± 2 | <0.05 | 40.5 ± 1.0 | 38.6 ± 0.8 | <0.0001 |

TABLE 6
Blood cardiometabolic endpoint values after 8 weeks of cookie intake.

| | Group A | | | Group B | | |
|---|---|---|---|---|---|---|
| | Placebo | | | Cacao Pericarp Flour | | |
| | BASAL | FINAL | P | BASAL | FINAL | P |
| Triglycerides | 207.8 ± 15.8 | 185.9 ± 22.4 | 0.20 | 204.2 ± 17.4 | 149.3 ± 12.1 | 0.003 |
| (mg/dL) | | | | | | |
| Glucose | 84 ± 2.3 | 80.7 ± 2.1 | 0.06 | 91 ± 3.5 | 81.09 ± 2.9 | 0.01 |
| (mg/dL) | | | | | | |
| Cholesterol | 178.5 ± 7.9 | 171.8 ± 10.9 | 0.18 | 217.8 ± 11.7 | 205.7 ± 14.7 | 0.18 |
| (mg/dL) | | | | | | |
| LDL (mg/dL) | 98.80 ± 7.5 | 97.70 ± 10.9 | 0.44 | 134.1 ± 9.7 | 130.2 ± 11.4 | 0.37 |
| HDL (mg/dL) | 38.1 ± 1.8 | 37 ± 2.2 | 0.26 | 42.6 ± 2.02 | 45.6 ± 3.6 | 0.17 |
| TG/HDL | 5.6 ± 0.5 | 5.4 ± 0.8 | 0.38 | 4.9 ± 0.4 | 3.4 ± 0.3 | 0.008 |
| LDL/HDL | 3.2 ± 0.2 | 3.3 ± 0.2 | 0.30 | 3.2 ± 0.3 | 2.9 ± 0.19 | 0.19 |

Physical results after 8 weeks of cookie supplementation are summarized in Table 5. Diet and exercise counseling induced favorable changes in both groups, but there are no differences between groups. Changes in cardiometabolic blood markers are summarized in Table 6. The consumption of placebo cookies induced no significant changes in any of markers evaluated. In contrast, consumption of the experimental (containing cacao pericarp flour) cookies induced positive and significant changes in triglycerides, glucose and TG/HDL ratio. HDL decreased slightly in placebo group and shows a non-significant increase in experimental group. These results indicate a decrease in cardiometabolic risk in subjects who consumed CP flour supplemented cookies. The experimental (cacao pericarp flour) group evidences a surprisingly significant decrease in blood triglycerides levels.

Left hand grip strength (non-dominant hand) can be evaluated before and after treatment with placebo or CP flour containing cookies with a dynamometer (Camry Grip Strength Dynamometer, City of Industry, Calif.). Subjects are instructed to apply as much force as they can. Test was repeated 3 times (30 seconds between tests), the highest result was recorded. Differences at the beginning of study and at the end (8 weeks) were compared in both groups.

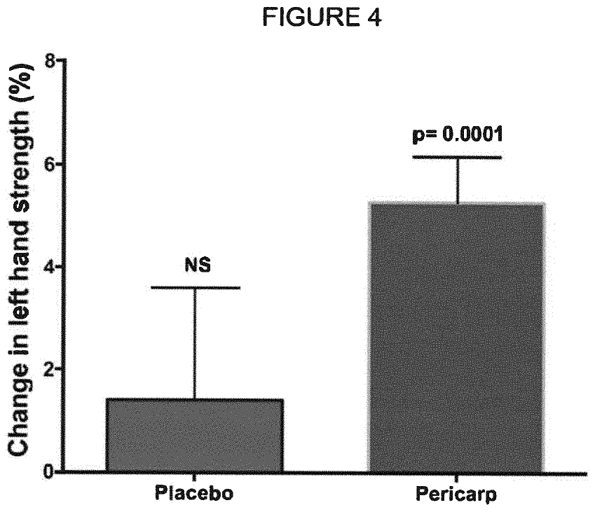

Subjects in the placebo group show no change in hand grip strength. However, the ingestion of CP flour cookies does induce a significant increase in hand grip strength (FIG. 4).

### EXAMPLE 4

The following pertains to the manufacturing and use of a topical cream using the cacao pericarp (CP) flour and proof of its beneficial effects on human skin. For this study, a control cream (control cream) was manufactured and compared with the same cream in which CP flour was added (CP cream). The basic cream ingredients are noted in the table below:

TABLE 7
Components of Exemplary Topical Cream (CP Cream).

| | QUANTITY |
|---|---|
| | (GRAMS UNLESS |
| | NOTED |
| COMPONENT | OTHERWISE) |
| GLYCERYL | 90 |
| MONOESTEREATE | |
| EUMULGIN C 700 | 45 |
| CETYL ALCOHOL | 45 |
| ANHYDROUS LANOLIN | 45 |
| CETIOL V | 120 |
| MYRITOL 318 | 90 |
| EUTANOL G | 150 |
| VEEGUM | 15 |
| SORBITOL | 75 |
| NIPAGIN | 9 |
| DISTILLED WATER | 675 (ML) |
| CP FLOUR (PRESENT | 0.1% |
| ONLY IN CP CREAM) | |

Experimental Set Up.

Analysis of cream effects on hand wrinkles and skin color. Subjects (n=22, 45-65 years old) of both genders were randomly assigned to either, a) control cream (n=10) or b) CP cream (n=12). Subjects were instructed to apply approximately 200 microliters of cream every morning on the back of the right hand and to extend it uniformly over the hand every day for a period of 4 weeks.

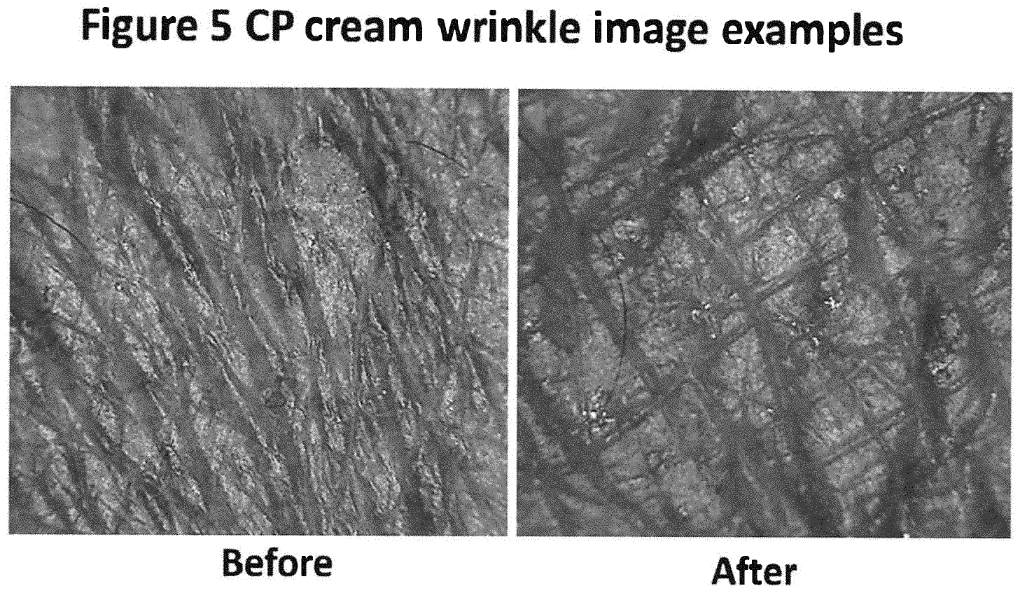

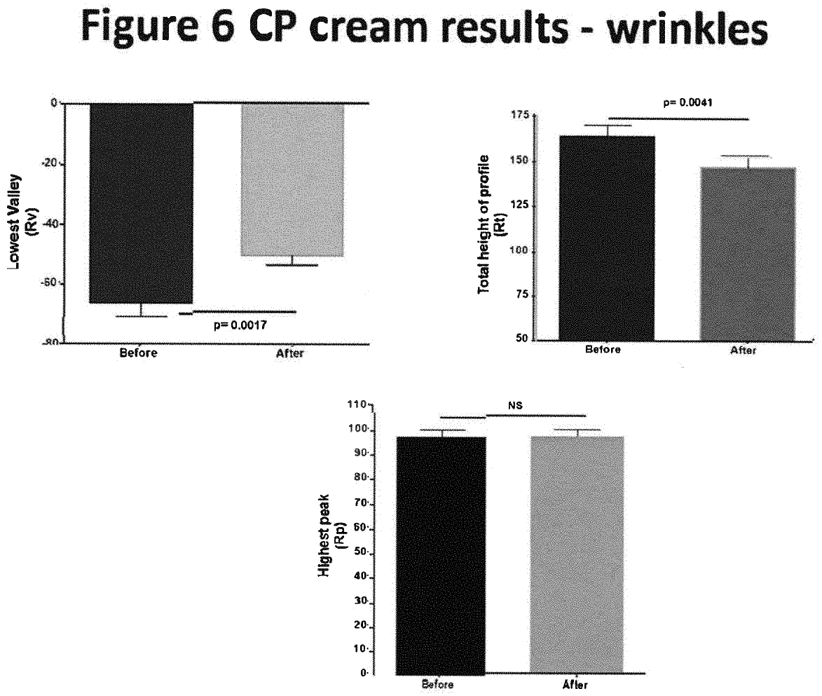

For hand wrinkle analysis, images were taken on the back of the hand using a cast to place the arm in the same uniform position while holding a 3 cm cylinder. Images were recorded using a Multifunction UV Skin Analysis System (Skin Scope Analyzer EH-2100) at approximately the same spot (middle part of the hand). The analysis of the images acquired was performed using image J computer software and the following wrinkle endpoints were generated: 1) total height of the wrinkle profile (Rt), 2) highest peak (Rp) and, 3) lowest valley (Rv). Changes in hand wrinkles were analyzed comparing Rt, Rp and Rv before and after 4 weeks of treatment using a paired t-test (statistically different if p<0.05). No statistical differences were detected in Rt, Rp and Rv with the control cream. As illustrated in FIG. 5 and summarized in FIG. 6 with the CP cream, significant decreases were detected in the lowest valley (Rv) and consequently, in the total height profile (Rt) of the wrinkles (i.e., a flatter skin profile develops).

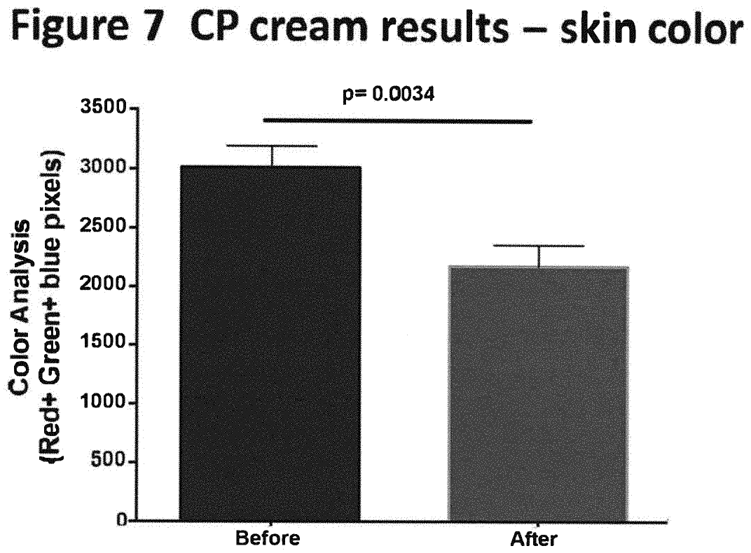

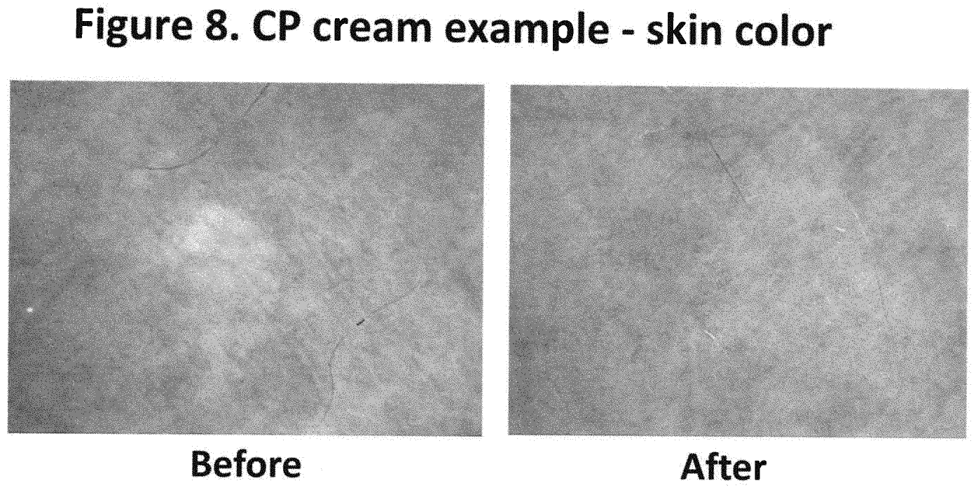

From the same images acquired, color changes were analyzed using a different image J software option (color inspector 3D plugin). As summarized in FIG. 7, the CP cream managed to reduce skin pigmentation to a lighter overall intensity. FIG. 8 shows example images of skin Before and After treatment with the CP cream and the reduction in discoloration uneven tone.

The terminology used herein is for the purpose of describing particular embodiments only and is not intended to be limiting of any method or approach. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The invention illustratively described here may be practiced in the absence of any element or elements, limitation or limitations which is not specifically disclosed herein. Thus, for example, in each instance herein any of the terms “comprising”, “consisting essentially of” and “consisting of” may be replaced with either of the other two terms.

The invention may be embodied in other specific forms without departing from the spirit or essential characteristics thereof. The present embodiments are therefore to be considered in all respects as illustrative and not restrictive, the scope of the invention being indicated by the claims of the application rather than by the foregoing description, and all changes which come within the meaning and range of equivalency of the claims are therefore intended to be embraced therein.

What is claimed is:

1. A method for the preparation of flour from cacao fruit comprising isolating pericarp material from cacao pods, wherein the pericarp material is free of cacao seeds and pulp, grinding the pericarp material to a paste, freeze drying the paste under vacuum pressure to a moisture level of less than 10%, and further grinding the dried paste to form a flour composition.

2. The method of claim 1, wherein the drying is maintained until the moisture level is 8% or less.

3. The method of claim 1, wherein the dried paste is ground until the average particle size is 0.500 mm or less.

4. The method of claim 2, wherein the dried paste is ground until the average particle size is 0.425 mm or less.

5. The method of claim 1, further comprising measuring the total polyphenol content of the pericarp material or the flour composition.

6. The method of claim 1, further comprising measuring the total flavonoid content of the pericarp material or the flour composition.

7. The method of claim 1, further comprising measuring the total antioxidant content of the pericarp material or the flour composition.

8. The method of claim 1, further comprising using the flour composition to prepare a food product.

9. A method for the preparation of flour from cacao pod husk comprising isolating pericarp material from cacao pods, wherein the pericarp material is free of cacao seeds and pulp, grinding the pericarp material to a paste, microwave drying the paste to a moisture level of less than 10%, and then grinding the dried pericarp material to form a flour composition.

10. The method of claim 9, wherein the drying is maintained until the moisture level is 8% or less.

11. The method of claim 9, wherein the dried paste is ground until the average particle size is 0.500 mm or less.

12. The method of claim 9, wherein the dried paste is ground until the average particle size is 0.425 mm or less.

13. The method of claim 9, further comprising measuring the total polyphenol content of the pericarp material or the flour composition.

14. The method of claim 9, further comprising measuring the total flavonoid content of the pericarp material or the flour composition.

15. The method of claim 9, further comprising measuring the total antioxidant content of the pericarp material or the flour composition.

16. The method of claim 9, further comprising using the flour composition to prepare a food product.

17. A prophylactic method of treating a human subject comprising providing a cacao pod husk ingredient derived only from the pericarp of the pod, and administering the ingredient daily at an amount that delivers one of: a total polyphenol content equivalent to about 12 mg of (-)-epicatechin per capsule; or equivalent to about 25 mg of (-)-epicatechin per dose twice a day; or equivalent to a total flavonoids content of 12.5 mg or greater per day; or equivalent to about total flavonoids of 12.5 mg or greater twice per day; or equivalent to a total flavonoids content of 25 mg or greater per day; or equivalent to about total flavonoids of 25 mg or greater twice per day.

18. The method of claim 17, further comprising monitoring one of more of the following cardiometabolic markers in the blood of the subject: triglyceride levels; LDL levels; glucose levels; LDL/HDL ratio, Body Mass Index, muscle mass, or triglyceride/HDL ratio.

19. A method of lowering blood triglyceride levels in a human subject with elevated levels of triglycerides comprising preparing a composition comprising an effective dose of a cacao pericarp flour or extract of cacao pericarp material, and orally administering the composition daily to the subject daily.

20. The method of claim 17, wherein the subject has triglycerides levels between 150-350 mg/dL prior to the treatment.

21. The method of claim 17, wherein the composition is administered prior to a meal.

22. The method of claim 17, wherein the composition is administered during a meal.

23. The method of claim 17, wherein the composition is administered between meals.

24. The method of claim 17, wherein the composition is administered daily prior to each meal.

25. The method of using of CP flour made from the method of claim 1, further comprising adding CP flour to food, beverage or supplements to lower triglycerides in humans.

26. The method of claim 25, further comprising adding CP flour to food, beverage or supplements to lower blood glucose in humans.

27. The method of claim 25, further comprising adding CP flour to food, beverage or supplements to decrease LDL/HDL ratios in humans.

28. The method of claim 25, further comprising adding CP flour to food, beverage or supplements to decrease Triglyceride/HDL ratios in humans.

29. The method of claim 25, further comprising adding CP flour to food, beverage or supplements to reduce waist circumference in humans.

30. The method of claim 25, further comprising adding CP flour to food, beverage or supplements to reduce Body Mass Index in humans.

31. The method of claim 25, further comprising adding CP flour to food, beverage or supplements to increase muscle mass in humans.

32. The method of claim 25, further comprising adding CP flour to food, beverage or supplements to increase muscle grip strength in humans.

33. A topical cosmetic composition comprising a CP flour composition and a cosmetically acceptable carrier.

34. The composition of claim 33, further comprising one or more of: (a) a source of bio-retinol (b) a source of retinol-like activity; (c) a source of sodium hyaluronic acid or other cosmetically active acid; (d) a source of moisturizing saccharide complex; (e) an ascorbic acid source; and (f) a source of anti-oxidant components.

35. A method of treating skin by topical administration of a CP flour comprising providing a composition of claim 33 and applying it to the skin of a patient at least once per day.

36. The method of claim 35, wherein the CP flour is present in the composition at a range from 0.1% to 1% by weight.
